(19) United States
(12) Patent Application Publication
(10) Pub. No.: US 2014/0020451 A1
(43) Pub. Date: Jan. 23, 2014

(54) FUEL PROPERTY SENSOR

(71) Applicant: DENSO CORPORATION, Kariya-city (JP)

(72) Inventors: Tatsuya KITANAKA, Nagoya-city (JP); Jun TARUI, Kariya-city (JP); Hiroshi NAKAMURA, Nishio-city (JP); Masato UENO, Takahama-city (JP); Dai OSADA, Kariya-city (JP); Sadahito FUKUMORI, Okazaki-city (JP)

(21) Appl. No.: 13/910,369

(22) Filed: Jun. 5, 2013

(30) Foreign Application Priority Data
Jul. 23, 2012 (JP) 2012-162529

(51) Int. Cl. G01N 33/22

(52) U.S. Cl. CPC G01N 33/22
USPC 73/53.01

(57) ABSTRACT

A fuel property sensor includes an electrode portion, a first thermistor, a second thermistor, and a circuit portion. The electrode portion detects an electrostatic capacity varied according to an ethanol concentration in fuel as a fuel characteristic. The first thermistor is disposed in a flowing area inside the electrode portion, where the fuel flows through. The second thermistor is disposed in a non-flowing area where no fuel flows through. The circuit portion which computes the ethanol concentration based on the electrostatic capacity, a first detection value detected by the first thermistor, and a second detection value detected by the second thermistor. Thus, a fuel temperature can be properly detected, and a detection accuracy of the fuel characteristic can be improved.

### CROSS-REFERENCE TO RELATED APPLICATION

This application is based on Japanese Patent Application No. 2012-162529 filed on Jul. 23, 2012, the disclosure of which is incorporated herein by reference.

### TECHNICAL FIELD

The present disclosure relates to a fuel property sensor which detects a fuel characteristic.

### BACKGROUND

Conventionally, an alcohol mixed gasoline, which is a low-pollution material, is used as a fuel in an engine of an automobile. The alcohol mixed gasoline is referred to as a mixed gasoline, hereinafter. An air-fuel ratio of the mixed gasoline may be changed due to an alcohol concentration in the fuel. Thus, it is necessary to measure the alcohol concentration for controlling the air-fuel ratio of the mixed gasoline to be equal to an optimal air-fuel ratio.

It is preferable that a physical constant having a high variation rate is used for accurately measuring a fuel characteristic such as the alcohol concentration of the mixed gasoline. The alcohol concentration of the mixed gasoline is measured based on an electrostatic capacity of an electrostatic capacity area. The electrostatic capacity area is formed by two electrodes and the fuel therebetween. JP-2011-107070A describes a technology which detects a fuel temperature and corrects the alcohol concentration with respect to the electrostatic capacity based on the fuel temperature, because the electrostatic capacity may be changed due to the fuel temperature.

However, a difference between an atmospheric temperature of a fuel property sensor and the fuel temperature may be large, or a circuit portion which computes the alcohol concentration may self-heat. In this case, a temperature sensor of JP-2011-107070A may incorrectly detect the fuel temperature, because a temperature variation causes due to an affect of the atmospheric temperature or an affect of a self-heating in the circuit portion. Therefore, the alcohol concentration in the fuel may be incorrectly measured.

### SUMMARY

It is an object of the present disclosure to provide a fuel property sensor which accurately detects a fuel characteristic.

According to an aspect of the present disclosure, the fuel property sensor includes an electrode portion, a first temperature sensor, a second temperature sensor, and a circuit portion.

The electrode portion disposed in a fuel space detects an electrical characteristic value which varies according to the fuel characteristic. The first temperature sensor is disposed in an area inside of the electrode portion where the fuel flows through. The second temperature sensor is disposed in an area where no fuel flows through. The circuit portion computes the fuel characteristic based on the electrical characteristic value detected by the electrode portion, a first detection value detected by the first temperature sensor, and a second detection value detected by the second temperature sensor.

A temperature distribution data about detection values of the first and second temperature sensors may be previously measured and stored in the circuit portion as a map. By using the map to compute the fuel temperature based on the detection values, the fuel temperature can be properly detected even though a temperature variation causes due to an affect of the atmospheric temperature of the fuel property sensor or the self-heating of the circuit portion. Therefore, a detection accuracy of the fuel characteristic can be improved.

### BRIEF DESCRIPTION OF THE DRAWINGS

The above and other objects and advantages of the present disclosure will be more readily apparent from the following detailed description made with reference to the accompanying drawings. In the drawings:

FIG. 1 is a schematic diagram showing an entire structure of a fuel supple system according to the present disclosure;

FIG. 2 is a sectional view showing a fuel property sensor according to a first embodiment of the present disclosure;

FIG. 3 is a graph showing a relationship between an electrostatic capacity, an ethanol concentration in fuel, and a fuel temperature, according to the first embodiment;

FIG. 4 is a sectional view showing a fuel property sensor according to a second embodiment of the present disclosure; and

FIG. 5 is a sectional view showing a fuel property sensor according to a third embodiment of the present disclosure.

### DETAILED DESCRIPTION

Hereafter, fuel property sensors according to the present disclosure will be described referring to the drawings. In embodiments of the present disclosure, a part that corresponds to a matter described in a preceding embodiment may be assigned with the same reference numeral, and redundant explanation for the part may be omitted. When only a part of a configuration is described in an embodiment, another preceding embodiment may be applied to the other parts of the configuration. The parts may be combined even if it is not explicitly described that the parts can be combined. The embodiments may be partially combined even if it is not explicitly described that the embodiments can be combined, provided there is no harm in the combination.

### FIRST EMBODIMENT

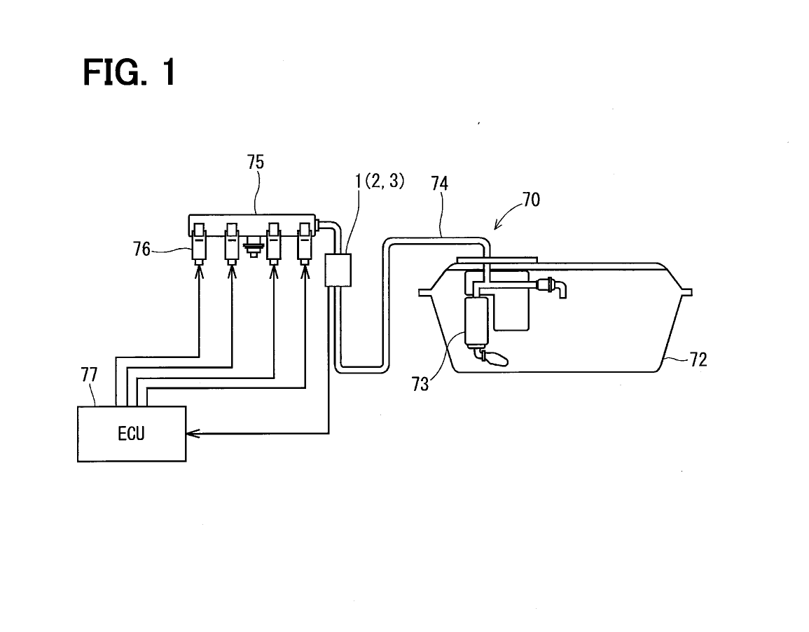

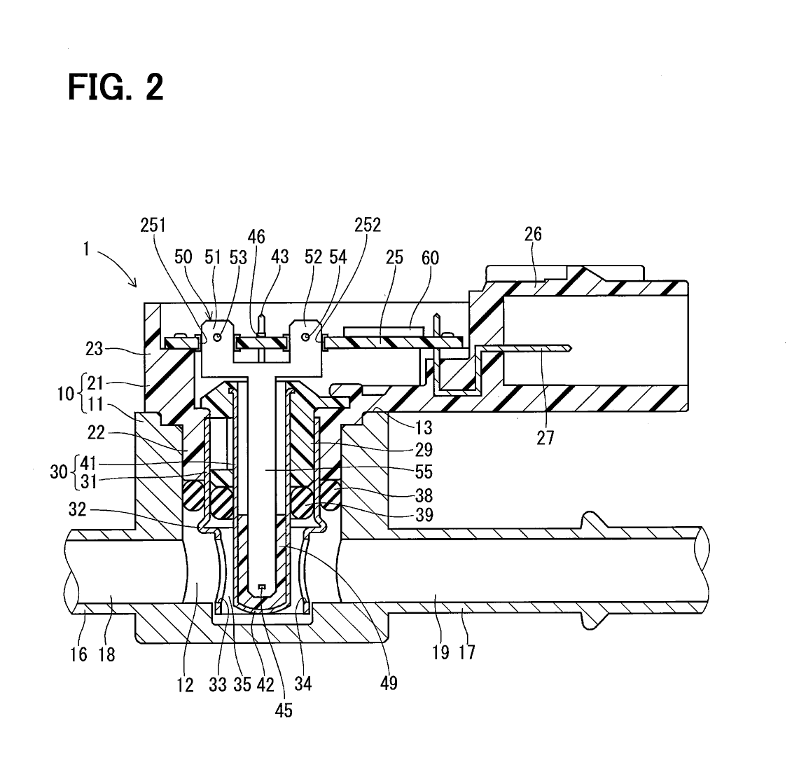

A fuel property sensor 1 according to a first embodiment of the present disclosure is shown in FIGS. 1 and 2.

As shown in FIG. 1, the fuel property sensor 1, which detects an alcohol concentration (e.g., ethanol concentration) in fuel as a fuel characteristic, is provided in a fuel supple system 70 of a vehicle. Specifically, the fuel property sensor 1 is provided in a fuel pipe 74 which is connected with a fuel tank 72 and a delivery pipe 75.

The fuel tank 72 accumulates a mixed gasoline (i.e., alcohol mixed gasoline) in which gasoline and alcohol (ethanol) are mixed. The mixed gasoline is referred to as a fuel hereafter. The fuel tank 72 can optionally refuel a mixed liquid of the gasoline and the ethanol, the gasoline, or the ethanol. Therefore, in the fuel tank 72, the ethanol concentration in fuel is in a range from zero to 100 percentages. The ethanol concentration may be varied when and after the fuel tank 72 refuels.

The fuel accumulated in the fuel tank 72 is press-sent by a fuel pump 73 to the delivery pipe 75 via the fuel pipe 74. Then, the fuel is injected by an injector 76 into an intake pipe (not shown) or a cylinder. The injector 76 is driven and controlled by an ECU 77 of an engine.

The ECU 77, which is configured of a microcomputer, is inputted by a detected signal of the fuel property sensor 1 and various detected signals relating to a driven of the engine. According to the first embodiment, the ECU 77 controls various control parameters such as an air-fuel ratio, a fuel injection quantity, or an ignition timing, according to the ethanol concentration in the fuel supplied to the engine so that the engine can operate in the optimal condition. In this case, the ethanol concentration is detected by the fuel property sensor 1, and the optimal condition may be a condition that toxic matter quantity of an exhaust gas is minimum and fuel consumption is reduced. It is preferable that the fuel property sensor 1 is provided at a position close to the injector 76, so that the engine is operated in the optimal condition by detecting the ethanol concentration at a position close enough to the injector 76.

As shown in FIG. 2, the fuel property sensor 1 includes a housing portion 10, an electrode portion 30, a first thermistor 45 as a first temperature sensor, a second thermistor 46 as a second temperature sensor, and a circuit portion 60.

The housing portion 10 includes a first housing 11 and a second housing 21 which are connected to each other.

The first housing 11 may be made of a stainless metal, and may be substantially cylindrical-shaped. The first housing 11 has therein a fuel chamber 12. A first connection pipe 16 and a second connection pipe 17 are provided at positions outside of the first housing 11 to extend in a radial direction of the first housing 11. The first and second connection pipes 16 and 17 may be made of the stainless metal, and may be substantially cylindrical-shaped. According to the first embodiment, the first and second connection pipes 16 and 17 are integrated with the first housing 11. The first connection pipe 16 has therein a first passage 18, and the second connection pipe 17 has therein a second passage 19. The first and second passages 18 and 19 communicate with the fuel chamber 12. The first and second connection pipes 16 and 17 are connected with the fuel pipe 74 shown in FIG. 1 via a connect member which is not shown. Thus, the fuel can be supplied to the first and second passages 18 and 19 and the fuel chamber 12.

The second housing 21 may be made of resin. The second housing 21 includes a cylinder portion 22 and a substrate accommodation portion 23.

The cylinder portion 22 is inserted into the first housing 11 from a housing opening 13. The housing opening 13 is provided at an end part of the first housing 11. A holder 29 is provided at a position inside of the cylinder portion 22 in a radial direction of the cylinder portion 22. The holder 29 may be made of resin, and may be substantially cylindrical-shaped. The holder 29 may be fixed to the second housing 21 by a fastener such as a thermal crimp.

The substrate accommodation portion 23 accommodates a circuit substrate 25 on which an electric circuit is printed. The circuit substrate 25 may be fixed to the second housing 21 by a fastener such as screw.

The substrate accommodation portion 23 includes a connector 26 which has a first terminal 27. A first end part of the first terminal 27 may be inserted into the circuit substrate 25 and electrically connected with the circuit substrate 25 by solder, for example. A middle part of the first terminal 27 is provided in the second housing 21. A second end part of the first terminal 27 is exposed to an interior of the connector 26. Thus, the connector 26 can be electrically connected with the ECU 77 shown in FIG. 1, a power source which is not shown, and the circuit portion 60.

The electrode portion 30 includes an outside electrode 31 and an inside electrode 41.

The outside electrode 31 and the inside electrode 41 may be substantially cylinder-shaped by press-processing a metal plate. According to the first embodiment, the outside and inside electrodes 31 and 41 are provided so as to be substantially concentric with each other. An end part of the outside electrode 31 close to the circuit substrate 25 may be connected with the circuit substrate 25 via a terminal which is not shown. An end part of the inside electrode 41 close to the circuit substrate 25 may be connected with the circuit substrate 25 via a second terminal 43. Hereafter, an end part of each component close to the circuit substrate 25 is referred to as a first side of the component, and an end part of each component opposite to the first side of the component is referred to as a second side of the component.

The outside electrode 31 is provided so that a first side of the outside electrode 31 is slidable in the cylinder portion 22. The first side of the outside electrode 31 is placed at a position between the cylinder portion 22 and the holder 29.

The outside electrode 31 includes a protrusion 32 protruding outward in a radial direction of the outside electrode 31. The protrusion 32 prevents a first O-ring 38 provided at a position between the outside electrode 31 and the first housing 11 from being taken away.

Thus, the first housing 11 and the outside electrode 31 are maintained at a predetermined distance therebetween and are electrically isolated from each other, because of the cylinder portion 22 and the first O-ring 38.

The inside electrode 41 is cylindrical-shaped and has a bottom portion 42 at a second side of the inside electrode 41. A first side of the inside electrode 41 is provided in the holder 29, so that the holder 29 is located at a position between the outside electrode 31 and the inside electrode 41. A second O-ring 39 is provided at a position between the outside electrode 31 and the inside electrode 41. Further, the second O-ring 39 is close to a second side of the holder 29.

Thus, the outside electrode 31 and the inside electrode 41 are maintained at a predetermined distance therebetween and are electrically isolated from each other, because of the holder 29 and the second O-ring 39.

The outside electrode 31 further includes a first fuel opening 33 and a second fuel opening 34 which are provided through a peripheral wall of the outside electrode 31 in the radial direction of the outside electrode 31. The fuel in the fuel chamber 12 flows into a space 35 between the outside electrode 31 and the inside electrode 41 via the first and second fuel openings 33 and 34. Thus, the outside electrode 31 and the inside electrode 41 may function as a condenser because the fuel flowing into the space 35 can function as a dielectric. That is, the electrode portion 30 detects an electrostatic capacity of the condenser.

Since the inside electrode 41 has the bottom portion 42, the fuel cannot flow into an interior of the inside electrode 41.

The first O-ring 38 is provided at a first position between the outside electrode 31 and the first housing 11. Further, the first position is close to a second side of the second housing 21 and a first side of the protrusion 32. Thus, the first O-ring 38 electrically isolates the outside electrode 31 and the first housing 11 from each other, and seals the first position therebetween.

The second O-ring 39 is provided at a second position between the outside electrode 31 and the inside electrode 41. Further, the second position is close to a second side of the holder 29. Thus, the second O-ring 39 electrically isolates the outside electrode 31 and the inside electrode 41 from each other, and seals the second position therebetween.

Thus, the first and second O-rings 38 and 39 prevent the fuel in the fuel chamber 12 and the space 35 from flowing into an area adjacent to first sides of the first and second O-rings 38 and 39. According to the first embodiment, an area adjacent to second sides of the first and second O-rings 38 and 39 may correspond to a flowing area where the fuel flows through, and the area adjacent to the first sides of the first and second O-rings 38 and 39 may correspond to a non-flowing area where no fuel flows through. Therefore, the circuit substrate 25 is disposed in the non-flowing area.

The first thermistor 45 is made of a resistor having a characteristic, and a resistance value of the resistor is varied according to temperature. The first thermistor 45 detects the resistance value as a first detection value. The first thermistor 45 which is chip-type is attached to a thermistor substrate 50. The thermistor substrate 50 may function as a supporting member to support the first thermistor 45. The first thermistor 45 is electrically connected with the circuit portion 60 via a first wiring (not shown) on the thermistor substrate 50.

The second thermistor 46 has the same configuration as the first thermistor 45, that is, the second thermistor 46 is made of a resistor having the same characteristic, and a resistance value of the resistor is varied according to temperature. The second thermistor 46 detects the resistance value as a second detection value. The second thermistor 46 which is chip-type is attached to the circuit substrate 25, and is electrically connected with the circuit portion 60. According to the first embodiment, the second thermistor 46 is placed at a position substantially coaxial with the first thermistor 45. Since the circuit substrate 25 is disposed in the non-flowing area, the second thermistor 46 is disposed in the non-flowing area.

The thermistor substrate 50 includes a first connection portion 51, a second connection portion 52, and an insertion portion 55.

The first and second connection portions 51 and 52 are inserted into a first through hole 251 and a second through hole 252, respectively. The first and second through holes 251 and 252 are provided in the circuit substrate 25. Further, the first and second connection portions 51 and 52 are fixed to the circuit substrate 25 by solder, for example. A third through hole 53 and a fourth through hole 54 are provided in the first and second connection portion 51 and 52, respectively. Further, the third through hole 53 is placed at a position so that at least a part of the third through hole 53 is in the first through hole 51, and the fourth through hole 54 is placed at a position so that at least a part of the fourth through hole 54 is in the second through hole 52. Thus, it is easy to fasten the above components. All of the edges and interior walls of the first, second, third and fourth through holes 53, 54, 251 and 252 are provided with electric conductors. Therefore, the thermistor substrate 50 is supported by and electrically connected with the circuit substrate 25.

The insertion portion 55 is inserted into the interior of the inside electrode 41. The first thermistor 45 is provided at a position close to a second side of the insertion portion 55. That is, the first thermistor 45 is disposed in the flowing area.

A first thermal conduction member 49 is provided between the insertion portion 55 and the inside electrode 41. Since a thermal conduction is executed rapidly by the first thermal conduction member 49, an error in a detection of the first thermistor 45 can be reduced even when the fuel temperature is varied. According to the first embodiment, a first side of the first thermal conduction member 49 is provided at a position close to the first and second O-ring 38 and 39.

The circuit portion 60 is made of a plurality of electric components provided on the circuit substrate 25. The circuit portion 60 computes the electrostatic capacity of the condenser configured by the fuel of the outside electrode 31, the inside electrode 41, or the space 35. In this case, the electrostatic capacity corresponds to an electrical characteristic value which is varied according to the fuel characteristic.

The circuit portion 60 acquires the first detection value detected by the first thermistor 45 and the second detection value detected by the second thermistor 46. According to the first embodiment, a temperature distribution data about the first detection value, the second detection value, and an actual fuel temperature, is previously measured and stored in the circuit portion 60 as a map. In the circuit portion 60, the fuel temperature is computed by a map calculation, based on the first detection value and the second detection value.

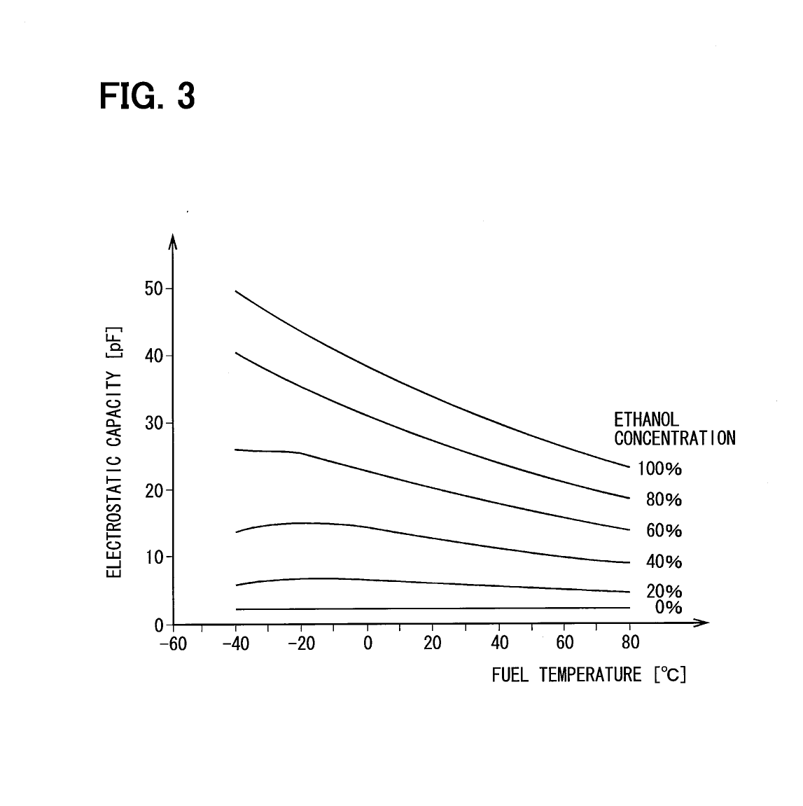

As shown in FIG. 3, the electrostatic capacity is varied according to the ethanol concentration and the fuel temperature. In the circuit portion 60, the ethanol concentration is computed by the relationship shown in FIG. 3, based on the electrostatic capacity and the fuel temperature.

As the above description, the fuel property sensor 1 according to the first embodiment includes the electrode portion 30, the first thermistor 45, the second thermistor 46, and the circuit portion 60.

The electrode portion 30 detects the electrostatic capacity varied according to the ethanol concentration which is a fuel characteristic. The first thermistor 45 is disposed in the flowing area inside of the electrode portion 30. The second thermistor 46 is disposed in the non-flowing area. The circuit portion 60 computes the ethanol concentration based on the electrostatic capacity, the first detection value, and the second detection value.

According to the first embodiment, the temperature distribution data is previously measured and stored in the circuit portion 60 as a map. Since the fuel temperature is computed by the map based on the first detection value and the second detection value, the fuel temperature can be properly detected even when a temperature distribution causes due to the atmospheric temperature of the fuel property sensor 1 or a self-heating of the circuit portion 60. Therefore, a detection accuracy of the ethanol concentration can be improved.

According to the first embodiment, the second thermistor 46 is attached to the circuit substrate 25. Even when a difference between a temperature of the circuit portion 60 and the fuel temperature is large due to the self-heating of the circuit portion 60, the fuel temperature can be properly detected. Therefore, the detection accuracy of the ethanol concentration can be improved.

### SECOND EMBODIMENT

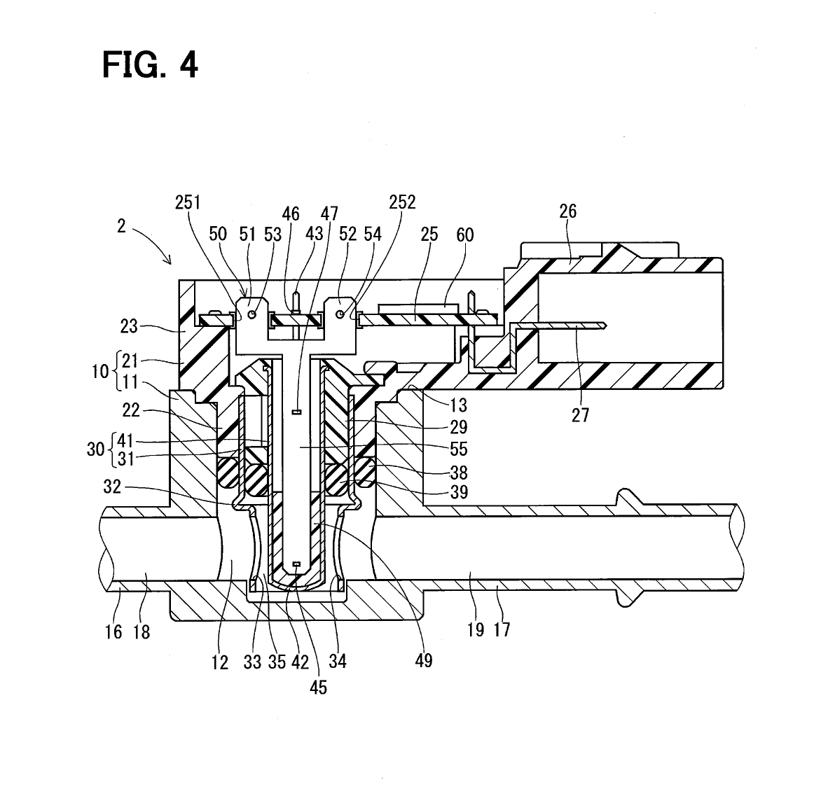

A fuel property sensor 2 according to a second embodiment of the present disclosure will be described referring to FIG. 4.

The fuel property sensor 2 has the same basic configuration as the fuel property sensor 1 of the first embodiment, and the description of the basic configuration will be omitted.

The fuel property sensor 2 further includes a third thermistor 47 as a third temperature sensor in addition of the first and second thermistors 45 and 46.

The third thermistor 47 has the same configuration as the first and second thermistors 45 and 46, that is, the third thermistor 47 is made of a resistor having the same characteristic, and a resistance value of the resistor is varied according to temperature. The third thermistor 47 detects the resistance value as a third detection value. The third thermistor 47 which is chip-type is attached to the thermistor substrate 50, and is electrically connected with the circuit portion 60 via a second wiring (not shown) on the thermistor substrate 50. It is preferable that the thermistor substrate 50 has a layered structure so that the first wiring connected with the first thermistor 45 and the circuit portion 60 and the second wiring connected with the third thermistor 47 and the circuit portion 60 are not crossed because the first and second wirings are placed at different layers.

The third thermistor 47 is disposed in the non-flowing area between the first thermistor 45 and the second thermistor 46. Further, the third thermistor 47 is placed at a position substantially concentric with the first thermistor 45 and the second thermistor 46.

According to the second embodiment, the temperature distribution data about the first detection value, the second detection value, the third detection value, and the actual fuel temperature, is previously measured and stored in the circuit portion 60 as a map. In the circuit portion 60, the fuel temperature is computed by the map calculation, based on the first detection value, the second detection value, and the third detection value.

According to the second embodiment, the fuel property sensor 2 further includes the third thermistor 47 provided between the first thermistor 45 and the second thermistor 46. The circuit portion 60 computes the ethanol concentration based on the electrostatic capacity, the first detection value, the second detection value, and the third detection value. Therefore, the second embodiment may have the same effects of the first embodiment. Further, according to the second embodiment, the temperature distribution measurement can be more accurate because the three thermistors 45 to 47 are provided. Thus, the fuel temperature can be accurately detected, and the detection accuracy of the ethanol concentration can be further improved.

For example, when a temperature detected by the third thermistor 47 is not in a range between a temperature detected by the first thermistor 45 and a temperature detected by the second thermistor 46, it can be determined that at least one of the three thermistors 45 to 47 is abnormal. Thus, an abnormality of the three thermistors 45 to 47 can be detected at an early stage.

When the abnormality is detected, it is preferable that the ECU 77 is noticed that the abnormality causes, and an engine control is switched to a fail mode.

According to the second embodiment, the third thermistor 47 is disposed in the non-flowing area. Thus, the fuel temperature can be properly detected even though a difference between the atmospheric temperature and the fuel temperature is large. Further, the detection accuracy of the ethanol concentration can be improved.

### THIRD EMBODIMENT

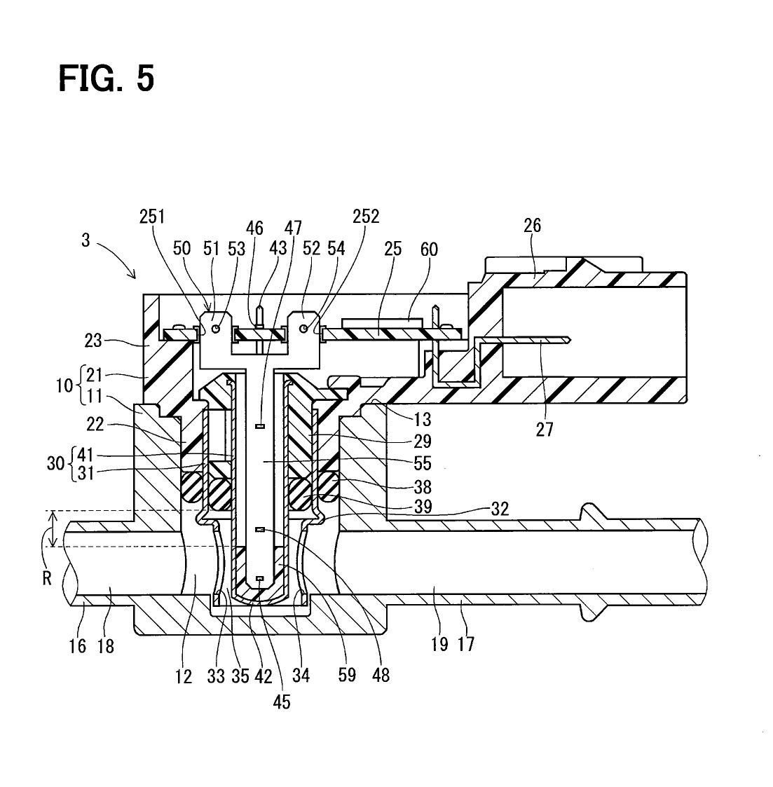

A fuel property sensor 3 according to a third embodiment of the present disclosure will be described referring to FIG. 5.

The fuel property sensor 3 has the same basic configuration as the fuel property sensor 2 of the second embodiment, and the description of the basic configuration will be omitted.

The fuel property sensor 3 further includes a fourth thermistor 48 in addition of the three thermistors 45 to 47. According to the third embodiment, both the third thermistor 47 and the fourth thermistor 48 may be used as third temperature sensors.

The fourth thermistor 48 has the same configuration as the three thermistors 45 to 47, that is, the fourth thermistor 48 is made of a resistor having the same characteristic, and a resistance value of the resistor is varied according to temperature. The fourth thermistor 48 detects the resistance value as a fourth detection value. According to the present disclosure, the third detection value may include the fourth detection value. The fourth thermistor 48 which is chip-type is attached to the thermistor substrate 50, and is electrically connected with the circuit portion 60 via a third wiring (not shown) on the thermistor substrate 50.

The fourth thermistor 48 is provided between the first thermistor 45 and the second thermistor 46 and provided between the first thermistor 45 and the third thermistor 47.

A second thermal conduction member 59 is provided between the thermistor substrate 50 and the electrode portion 30. A first side of the second thermal conduction member 59 is closer to a second side of itself than that of the first thermal conduction member 49 is. Thus, an area R is provided as shown in FIG. 5, where no fuel flows through and the second thermal conduction member 59 is not provided. The fourth thermistor 48 is disposed in the area R which is a part of the flowing area.

According to the third embodiment, the temperature distribution data about the first detection value, the second detection value, the third detection value, the fourth detection value, and the actual fuel temperature, is previously measured and stored in the circuit portion 60 as a map. In the circuit portion 60, the fuel temperature is computed by the map calculation, based on the first detection value, the second detection value, the third detection value, and the fourth detection value.

Therefore, the third embodiment may have the same effects of the above embodiments.

According to the third embodiment, both the third thermistor 47 and the fourth thermistor 48 are provided as the third temperature sensors. In other words, at least one of the third temperature sensors may be provided between the first thermistor 45 and the second thermistor 46.

Further, according to the third embodiment, the temperature distribution measurement can be more accurate because at least four thermistors are provided. Thus, the fuel temperature can be accurately detected, and the detection accuracy of the ethanol concentration can be improved.

According to the third embodiment, one of the thermistors 45 to 48 which is abnormal can be identified by a majority decision based on a temperature distribution of the thermistors 45 to 48, because the thermistors 45 to 48 are provided at four positions. For example, when one of the first thermistor 45, the second thermistor 46, the third thermistor 47, or the fourth thermistor 48 is abnormal, the fuel temperature can be continuously measured by using the detection values of the thermistors except the abnormal thermistor. Thus, the ethanol concentration can be continuously measured. The fuel property sensor 3 may be configured such that the ECU 77 is noticed that one of the thermistors is abnormal.

Particularly, when the first thermistor 45 is abnormal, it is possible that a measuring error of the fuel temperature is increased. It is preferable that the measurement of the fuel temperature and the measurement of the ethanol concentration are terminated. Further, it is preferable that the ECU 77 is noticed that the abnormality of the first thermistor 45 causes, and the engine control is switched to a fail mode.

### OTHER EMBODIMENT

According to the above embodiments, the second thermistor 46 is attached to the circuit substrate 25 and is placed at a position substantially concentric with the first thermistor 45. However, the second thermistor 46 can be provided at any position in the non-flowing area. For example, the second thermistor 46 may be placed at any position on the circuit substrate 25. Alternatively, the second thermistor 46 may be placed at a position of the thermistor substrate 50 in the non-flowing area. Further, considering the self-heating of the circuit portion 60, it is preferable that the second thermistor 46 is placed at a position close to the circuit substrate 25. For example, the second thermistor 46 may be placed at a position of the thermistor substrate 50 close to the circuit substrate 25.

According to the above embodiments, the third thermistor 47 is disposed in the non-flowing area between the first thermistor 45 and the second thermistor 46. However, the third thermistor 47 may be provided at any position between the first thermistor 45 and the second thermistor 46. Further, in the third embodiment, the third thermistor 47 may be omitted so that the single fourth thermistor 48 is functioned as the third temperature sensor. This configuration may have the same effects of the above embodiments. In this case, the second thermal conduction member 59 may be configured as the same as the first thermal conduction member 49, that is, the first side of the second thermal conduction member 59 is provided at a position adjacent to the first and second O-rings 38 and 39. Alternatively, the second thermal conduction member 59 may be omitted.

A number of the third temperature sensors provided between the first temperature sensor and the second temperature sensor may be three or more. The fuel temperature can be measured more properly in accordance with an increase in number of the temperature sensors. A detection accuracy of the fuel temperature can be ensured when an abnormality causes in the temperature sensors.

According to the above embodiments, each thermistor is chip-type. However, the thermistor may be a thermistor other than that of chip-type. Then, the thermistor may be supported by a thermistor lead instead of being provided on a substrate. Further, the first temperature sensor, the second temperature sensor, and the third temperature sensor(s) may be different types from each other.

Furthermore, a device other than the thermistor may be used as a temperature sensor.

According to the above embodiments, each O-ring is used as a seal member to prevent the first side of the electrode portion from flowing through by fuel. However, the seal member may be a member which can introduce the fuel to the first side of the electrode portion other than the O-ring. For example, a glass seal may be used. In this case, an area which is adjacent to the part of the seal member may correspond to the flowing area, and an area which is adjacent to the first side of the seal member may correspond to the non-flowing area.

According to the above embodiments, the fuel property sensor is provided in the fuel pipe. However, the fuel property sensor may be provided in other places such as the fuel tank, as long as the part of the electrode portion can be disposed in a fuel space.

According to the above embodiments, the electrode portion is made of the outside electrode and the inside electrode which are substantially cylindrical-shaped. However, the electrode portion may be provided as any shape. Further, the fuel property sensor is not limited to detect the fuel characteristic based on the electrostatic capacity between the electrodes. The fuel property sensor may detect the fuel characteristic based on other electrical characteristic values between the electrodes, such as a resistor.

Furthermore, the fuel property sensor is not limited to detect the alcohol concentration in fuel. The fuel property sensor may detect other characteristics in fuel, such as an oxidation state.

While the present disclosure has been described with reference to the embodiments thereof, it is to be understood that the disclosure is not limited to the embodiments and constructions. The present disclosure is intended to cover various modification and equivalent arrangements. In addition, while the various combinations and configurations, which are preferred, other combinations and configurations, including more, less or only a single element, are also within the spirit and scope of the present disclosure.

What is claimed is:

1. A fuel property sensor comprising:
an electrode portion disposed in a fuel space to detect an electrical characteristic value varied according to a fuel characteristic;
a first temperature sensor disposed in a flowing area inside of the electrode portion, where the fuel flows through;
a second temperature sensor disposed in a non-flowing area where no fuel flows through; and
a circuit portion which computes the fuel characteristic based on the electrical characteristic value detected by the electrode portion, a first detection value detected by the first temperature sensor, and a second detection value detected by the second temperature sensor.

2. A fuel property sensor according to claim 1, wherein
the second temperature sensor is attached to a circuit substrate having the circuit portion.

3. A fuel property sensor according to claim 1, further comprising
at least one third temperature sensor disposed in an area between the first temperature sensor and the second temperature sensor, wherein
the circuit portion computes the fuel characteristic based on the electrical characteristic value detected by the electrode portion, the first detection value detected by the first temperature sensor, the second detection value detected by the second temperature sensor, and a third detection value detected by the third temperature sensor.

4. A fuel property sensor according to claim 3, wherein
the third temperature sensor is disposed in the non-flowing area.

5. A fuel property sensor according to claim 3, further comprising
a thermal conduction member provided between a supporting member supporting the first temperature sensor and the electrode portion, wherein
the third temperature sensor is disposed in a part of the flowing area, where the thermal conduction member is not provided.

6. A fuel property sensor according to claim 3, further comprising:
a plurality of the third temperature sensors disposed in the area between the first temperature sensor and the second temperature sensor; and
a thermal conduction member provided between a supporting member supporting the first temperature sensor and the electrode portion, wherein
at least one of the third temperature sensors is disposed in the non-flowing area, and
at least one of the third temperature sensors is disposed in a part of the flowing area, where the thermal conduction member is not provided.
